(19) United States
(12) Patent Application Publication
(10) Pub. No.: US 2016/0089255 A1
(43) Pub. Date: Mar. 31, 2016

(54) REMOVABLE VASCULAR OCCLUSION DEVICE

(71) Applicant: ANAXIOM CORPORATION, Laguna Hills, CA (US)

(72) Inventors: Olav Bergheim, Laguna Hills, CA (US); William Cohn, Houston, TX (US); Reynolds Delgado, Houston, TX (US); Timothy Murphy, Providence, RI (US); Chris Swain, Austin, TX (US)

(21) Appl. No.: 14/862,592

(22) Filed: Sep. 23, 2015

Related U.S. Application Data
(60) Provisional application No. 62/056,215, filed on Sep. 26, 2014.

(51) Int. Cl. A61F 5/00; A61M 29/02

(52) U.S. Cl. CPC A61F 5/003; A61M 29/02

(57) ABSTRACT

Disclosed herein are embodiments of an occlusion device that can be delivered into the vascular system of a patient for inducing weight loss. The device can be formed from a body that can expand from a collapsed to an expanded position, where the body can be configured to at least partially occlude a blood vessel. Further, the device can comprise a first and second end joined by a center section. One of the ends can contain a retrieval element for easily removing the device from the patient.

### INCORPORATION BY REFERENCE TO ANY PRIORITY APPLICATIONS

This Application claims from the benefit of U.S. Provisional Application No. 62/056,215, filed Sep. 26, 2014, titled “REMOVABLE VASCULAR OCCLUSION DEVICE,” the entirety of which is incorporated herein by reference.

### BACKGROUND

1. Field

Disclosed herein are embodiments of an occlusion device which can be deployed in a body lumen thereby occluding the body lumen, and later removed, and methods of using the occlusion device for inducing weight loss.

2. Description of the Related Art

Obesity is a serious medical condition. Complications associated with obesity include hypertension, diabetes, coronary artery disease, stroke, congestive heart failure, multiple orthopedic problems, pulmonary insufficiency, etc. Obesity can significantly affect quality of life and can result in a markedly decreased life expectancy.

To date, surgery is the only proven method for inducing substantial long-term weight loss in a patient. Numerous surgical procedures and devices have been developed to induce such weight loss, e.g., “stomach stapling”, the Roux-en-Y (“The Roux”) bypass procedure, the vertical banded gastroplasty (“VBG”) procedure, etc. However, all of the known surgical procedures and devices developed to date for inducing weight loss in a patient suffer from one or more significant disadvantages.

### SUMMARY

Disclosed herein are embodiments of an occlusion device comprising a body configured to expand from a collapsed to an expanded position, wherein the body is configured to at least partially occlude a portion of a blood vessel, the body comprising a first end, a second end located generally opposite the first end, and a center section connecting the first and second ends, and a retrieval element attached to the body at the first end.

In some embodiments, the second end can be closed. In some embodiments, the second end can be open.

In some embodiments, the retrieval element can comprise a hook. In some embodiments, the retrieval element can comprise at least one loop. In some embodiments, the retrieval element can comprise at least one expandable finger.

In some embodiments, the body can be a silicone balloon. In some embodiments, the body can be a mesh plug. In some embodiments, the body can be a stent.

In some embodiments, the device can further comprise a covering configured to at least partially surround the body. In some embodiments, the covering can comprise PTFE, FEP, very high density ePTFE, or silicone.

Also disclosed herein are embodiments of an occlusion system comprising an occlusion device comprising a body configured to expand from a collapsed to an expanded position, wherein the body is configured to at least partially occlude a portion of a vasculature system, the body comprising a first end, a second end located generally opposite the first end, and a center section connecting the first and second ends, and a retrieval element attached to the body at the first end, a re-capture device configured engage with the retrieval element of the occlusion device, and a sheath, the sheath configured to retain the re-capture device and the occlusion device in the collapsed position.

Also disclosed herein are embodiments of a method of using a device comprising inserting an occlusion device into a patient, the occlusion device comprising a body configured to expand from a collapsed to an expanded position, wherein the body is configured to at least partially occlude a portion of a vasculature system, the body comprising a first end, a second end located generally opposite the first end, and a center section connecting the first and second ends, and a retrieval element attached to the body at the first end, expanding the occlusion device into the expanded position, inserting a recovery element into the patient, the recovery element configured to engage with the retrieval element of the occlusion device, and removing the recovery element and the occlusion device from the patient.

In some embodiments, expanding the occlusion device can occur automatically when the occlusion device is located in a desired position. In some embodiments, inserting the recovery element can comprise inserting the recovery element at least partially surrounded by a second sheath. In some embodiments, removing the recovery element and the occlusion device can comprise the occlusion device being at least partially surrounded by the second sheath.

Disclosed herein are embodiments of an occlusion device for reversibly occluding a body lumen, the occlusion device comprising an elongate member, comprising first and second end portions, wherein the elongate member is configured to reversibly expand from a collapsed state to an expanded state at a site within the body lumen, and wherein the elongate member is further configured to at least partially occlude the body lumen at the site, and a retrieval element coupled to the elongate member along one of the first or second end portions and extending generally outward from the elongate member into the body lumen.

In some embodiments, one or both of the first and second end portions can be closed. In some embodiments, the body lumen can be a blood vessel. In some embodiments, the retrieval element can comprise a hook. In some embodiments, the retrieval element can comprise at least one loop. In some embodiments, the retrieval element can comprise at least one expandable finger. In some embodiments, the elongate member can comprise a silicone balloon. In some embodiments, the elongate member can comprise a mesh plug. In some embodiments, the elongate member can comprise a stent.

In some embodiments, the occlusion device can further comprise a covering configured to at least partially surround the elongate member. In some embodiments, the covering can comprise PTFE, FEP, very high density ePTFE, or silicone. In some embodiments, the occlusion device can further comprise at least one anchoring element configured to extend radially outward from the elongate member toward a wall of the body lumen and engage the wall.

Also disclosed herein are embodiments of an occlusion system comprising an occlusion device comprising an elongate member, comprising first and second end portions, wherein the elongate member is configured to reversibly expand from a collapsed state to an expanded state at a site within a body lumen, and wherein the elongate member is further configured to at least partially occlude the body lumen at the site, and a retrieval element coupled to the elongate member along one of the first or second end portions and extending generally outward from the elongate member into the body lumen, a re-capture device configured to engage the retrieval element of the occlusion device, and a sheath configured to surround the re-capture device together with the engaged occlusion device, wherein the elongate member of the occlusion device is retained in the collapsed state within the sheath.

Also disclosed herein are embodiments of a method of reversibly occluding a blood vessel for a period of time, the method comprising deploying an occlusion device at a site within the blood vessel of a patient, the occlusion device comprising an elongate member, comprising first and second end portions, wherein the elongate member is configured to reversibly expand from a collapsed state to an expanded state at the site within the blood vessel, and wherein the elongate member is further configured to occlude the blood vessel at the site, and a retrieval element coupled to the elongate member along one of the first or second end portions and extending generally outward from the elongate member into a lumen of the blood vessel, expanding the elongate member, thereby occluding the blood vessel at the site, collapsing the elongate member after the period of time has passed, deploying a re-capture device at the site within the blood vessel, wherein the re-capture device is configured to engage the retrieval element, engaging the retrieval element, co-axially engaging the re-capture device together with the engaged occlusion device within a sheath, and removing the sheath with the re-capture device and engaged occlusion device from the patient.

In some embodiments, the occlusion device can self-expand when deployed at the site. In some embodiments, the deploying the re-capture device can comprise inserting the re-capture device at least partially surrounded by a second sheath. In some embodiments, the collapsing can occur when the elongate member is pulled into the sheath. In some embodiments, the collapsing can occur by puncturing the elongate member.

Also disclosed herein are embodiments of a method of inducing weight loss, the method comprising deploying an occlusion device at a site within a target blood vessel that supplies blood to an organ of the gastrointestinal tract of a patient, the occlusion device comprising an elongate member, comprising first and second end portions, wherein the elongate member is configured to reversibly expand from a collapsed state to an expanded state at the site, and wherein the elongate member is further configured to at least partially occlude the target blood vessel at the site, and a retrieval element coupled to the elongate member along one of the first or second end portions and extending generally outward from the elongate member into a lumen of the target blood vessel, and expanding the elongate member, thereby at least partially occluding the target blood vessel, restricting blood flow to the organ, and inducing weight loss.

In some embodiments, the target blood vessel can be a celiac artery. In some embodiments, the target blood vessel can be selected from the group consisting of the left gastric artery, the right gastric artery, the left gastroepiploic artery, the right gastroepiploic artery, or the common hepatic artery.

### BRIEF DESCRIPTION OF THE DRAWINGS

FIGS. 1A-C illustrate embodiments of a vascular plug with a hook style removal feature configured as an occlusion device.

FIGS. 2A-D illustrate an embodiment of a closed stent with a hook style removal feature configured as an occlusion device.

FIGS. 3A-B illustrate an embodiment of an open stent with a hook style removal feature configured as an occlusion device.

FIG. 4 illustrates an embodiment of a snare for recapture of an embodiment of an occlusion device.

FIGS. 5A-C illustrate an embodiment of a wire loop configuration for use as a removal feature.

FIGS. 6A-C illustrate the geometry of an embodiment of an occlusion device.

FIGS. 7A-C illustrate an embodiment of a method for removing an occlusion device.

FIGS. 8A-E illustrate embodiments of an occlusion device in different stages of use, as well as a balloon based-occlusion device.

FIG. 9 illustrates an embodiment of an occlusion device having anchoring elements.

### DETAILED DESCRIPTION

Disclosed herein are embodiments of a system which can be used in a percutaneous vascular procedure in a patient, specifically for occlusion of a body lumen such as a blood vessel (e.g., artery) and its subsequent removal from the patient. In some embodiments, the disclosed occlusion device can be used in any body lumen, including e.g., gastrointestinal system ducts (cystic ducts and other bile ducts), ducts of the urogenital system, lymph ducts, mammary ducts, etc. In particular, the system can include a catheter assembly allowing for delivery, location, and placement of an occlusion device at a predetermined site. The occlusion device can have an elongate member attached to a retrieval element. The system can further include a device for capturing (or re-capturing) and removing the occlusion device placed within the body lumen. Thus, embodiments of the disclosed system can provide the ability to acutely occlude a blood vessel and eventually remove the occlusion from the blood vessel. In some embodiments, the degree of occlusion can also be varied during the treatment period, such that for example, substantially total occlusion can be followed by gradual (progressive or step-wise) reduction in the degree of occlusion.

Thus, the present disclosure provides a novel method and apparatus for inducing weight loss in a patient by intentionally occluding blood vessels in a patient, such as the celiac artery. The present disclosure also provides a novel method and apparatus for inducing weight loss in a patient by intentionally occluding a blood vessel other than the celiac artery, and/or in addition to the celiac artery. Further, the present disclosure provides a novel method and apparatus for treating a patient for purposes other than inducing weight loss in the patient by intentionally occluding a blood vessel. Moreover, the present disclosure provides a novel method and apparatus for removing an occlusion device after use.

In some embodiments, weight loss can be induced in a patient by intentionally occluding the celiac artery in order to create hypoperfusion in the stomach, whereby to induce weight loss in the patient. More particularly, the celiac artery supplies oxygenated blood to the stomach, liver, pancreas, spleen and to the superior half of the duodenum. The celiac artery is a major source of blood for the stomach, inasmuch as the other blood vessels supplying nourishment to the stomach provide adequate flow to maintain viability, but cannot provide the marked increase in blood flow seen in the post-prandial period.

Accordingly, in some embodiments, the celiac artery can be intentionally reversibly occluded gradually over time in order to create hypoperfusion in the stomach (and/or other gastrointestinal organs serviced by the celiac artery) so as to interfere with normal gastrointestinal function and thereby induce weight loss in the patient. Occlusion is preferably effected at a location where it will only interfere with gastrointestinal function and will not seriously impede other essential anatomical functions. By way of example but not limitation, occlusion may be effected in the trunk of the celiac artery. Alternatively, occlusion may be effected in branches of the celiac artery (e.g., the left or right gastric arteries, the left or right gastroepiploic arteries, the common hepatic artery, and/or other celiac artery branches).

Furthermore, and also in accordance with the present disclosure, occlusion may be intentionally induced in other blood vessels servicing the organs of the gastrointestinal tract, whereby to impede normal gastrointestinal function and thereby induce weight loss in the patient. By way of example but not limitation, occlusion may be intentionally induced in other mesenteric vessels (e.g., the superior mesenteric artery and/or its major branches), and/or mesenteric veins, etc.

In essence, the present disclosure discloses systems for the intentional occlusion of substantially any blood vessel servicing the gastrointestinal tract such that the occlusion diminishes normal gastrointestinal function, whereby to induce weight loss in the patient. In some embodiments, the intentional occlusion of the celiac artery (including one or more of its branches) can be performed so as to diminish gastrointestinal function and thereby induce weight loss in the patient.

Embodiments of a removable occlusion device disclosed herein can be used to occlude a blood vessel or artery. In some embodiments, the occlusion device can partially, or fully occlude, a vessel or artery for a specified period of time, wherein the occlusion device can then be removed. In some embodiments, the occlusion device can have a collapsed and expanded position, as well as any intermediate positions, allowing for the occlusion device to be easily inserted into the venous system of a patient in the collapsed position and expanded to the expanded position within the patient. Expansion and collapse can occur relatively quickly, such as in a matter of hours, minutes, or seconds. Further, in some embodiments, struts on the occlusion device can be oriented axially, and may not cross, allowing for ease of removal of the device as this configuration allows for ingrowth not to impede removal.

While certain embodiments are disclosed below, the occlusion device can be formed from any number of configurations, and can include a combination of components from the below description. For example, the occlusion device can be a braided stent, a self-expanding stent, a balloon expanding stent, an expanded wire, or a silicone balloon, among others. The occlusion device can be self-expanding or can be expanded through the use of another force. For example, if a balloon is used, the balloon can be filled with fluid or gas in order to expand the balloon.

### Occlusion Device Configurations

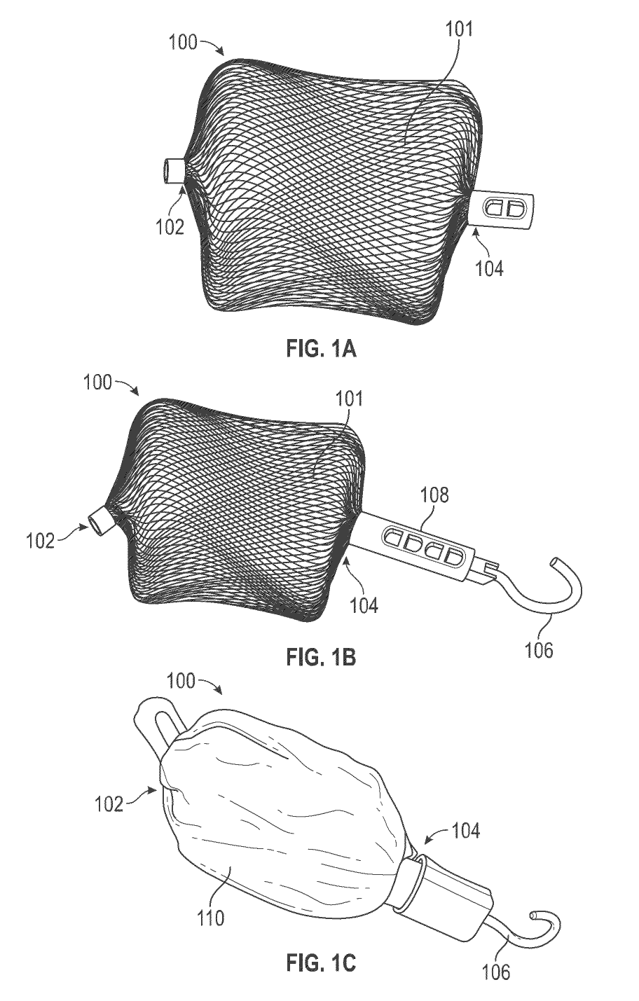

FIGS. 1A-C show an embodiment of an occlusion device 100 which can be used for inducing weight loss. As shown in FIG. 1A, the occlusion device 100 can be sized and shaped like a plug (e.g., having a generally cylindrical or tubular shape or elongate member), thought he particular shape is not limiting. In some embodiments, the occlusion device 100 can be formed of a biocompatible material, or can be coated with a biocompatible material to prevent or reduce any immune response.

As shown in FIGS. 1A-B, in some embodiments a body 101 of the device 100 may be made of a mesh or plurality of struts. The mesh can be small enough to prevent or slow blood from flowing through the body 101. The mesh can allow the body 101 to be flexible within a blood vessel in order to stay with the contours of a blood vessel.

The occlusion device 100 can be closed, crimped, or collapsed at one end 102, thereby at least partially occluding the vessel that the occlusion device 100 can be located in. In some embodiments, both ends 102/104 can be closed (or collapsed), as shown in FIG. 1A. The closure can be performed by, for example, crimping, welding, or bonding with adhesive, though the method of closure is not limiting and other methods can be used as well. In some embodiments, the occlusion device 100 can be self-expanding. In some embodiments, the device 100 can be formed from nitinol. Additional materials which can be used to form embodiments of the occlusion device are, for example stainless steel, cobalt-chromium, tantalum, or a polymer such as silicone or biodegradable/bioabsorbable material.

As shown in FIG. 1B, the occlusion device 100 can have an engagement element, such as hook 106, that can be used to help with withdrawal of the occlusion device 100 after use. The hook 106 can extend from either end 102/104 of the occlusion device 100. In some embodiments, a hook 106 can extend from both ends 102/104. The hook 106 can be made of a biocompatible material.

In some embodiments, the hook 106 can extend away from the device 100 at approximately the same length as the device 100. In some embodiments, the hook 106 can extend directly from the device 100 along a longitudinal axis of the device 100. In some embodiments, the hook 106 can be flexible, which can help prevent damage to the blood vessels. In some embodiments, the hook 106 can be rigid. In some embodiments, the hook 106 can be a fixed wire hook or a flexible nitinol hook. In some embodiments, the hook 106 can contain a flexible attachment 108 to the body 101 of the device 100. In some embodiments, the attachment can be rigid. The type of attachment is not limiting.

FIG. 1C shows an embodiment of an occlusion device 100 that is wrapped in a covering 110. In some embodiments, the occlusion device 100 can be partially or fully surrounded by the covering 110. In some embodiments, the covering can be used to prevent ingrowth of the blood vessel through the device, which allows for easier removal after the device has been implanted in a patient. Further, the covering can be used to help the occlusion of the blood vessel by not allowing blood to pass through the covering. In some embodiments, the covering can be, for example, PTFE, FEP, very high density ePTFE, silicone, or other similar material.

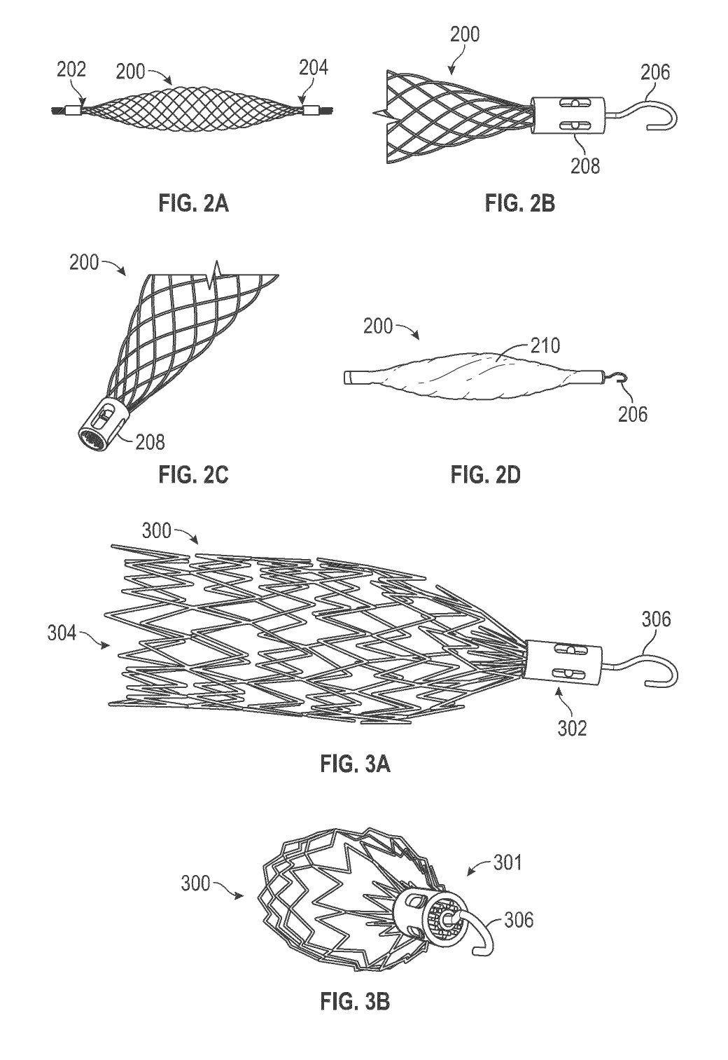

FIGS. 2A-C show an embodiment of an occlusion device 200. As shown in FIG. 2A, the occlusion device 200 can be similar in size and shape to a stent, and can be formed of a biocompatible material. As shown, the struts of the device 200 can be generally axially aligned. The occlusion device 200 can be closed at one end 202, thereby at least partially occluding the vessel that the occlusion device 200 can be located in. In some embodiments, both ends 202/204 can be closed.

As shown in FIG. 2B, the occlusion device 200 can have an engagement element, in this figure a hook 206, that can be used to help with withdrawal of the occlusion device 200 after use. The hook 206 can extend from either end 202/204 of the occlusion device 200. The hook 206 can be made of a biocompatible material. In some embodiments, the hook 206 can extend away from the device 200 at approximately the same length as the device 200. In some embodiments, the hook 206 can extend directly from the device 200. In some embodiments, the hook 206 can be flexible. In some embodiments, the hook 206 can be rigid. In some embodiments, the hook 206 can contain an attachment 208 to the device 200. In some embodiments, the attachment can be flexible or rigid. The type of attachment is not limiting. FIG. 2C shows the device 200 end without a hook 206, but with the attachment 208.

FIG. 2D shows an embodiment of an occlusion device 200 that is wrapped in a covering 210. The covering 210 can be used to further occlude a blood vessel, thus preventing blood from passing down the vessel. The covering 210 can be similar, or the same, as the covering 110 discussed above with respect to FIG. 1C.

FIGS. 3A-B show an embodiment of an occlusion device 300. As shown in FIG. 3A-B, the occlusion device 300 can be similar in size and shape to a stent (such as shown above with respect to FIGS. 2A-D), and can be formed of a biocompatible material. The occlusion device 300 can be closed at one end 302, thereby at least partially occluding the vessel that the occlusion device 300 can be located in. As shown, one end 304 of the device 300 can be open. In some embodiments, the occlusion device 300 can be self-expanding. In some embodiments, the device 300 can be formed from nitinol. Additional materials which can be used to form embodiments of the occlusion device are, for example stainless steel, cobalt-chromium, tantalum, or a polymer such as silicone or biodegradable/bioabsorbable material. Similar to the discussion above, the device 300 can have a hook 306. In some embodiments, similar to as discussed above, the occlusion device 300 can also have a cover surrounding the device 300 (not shown).

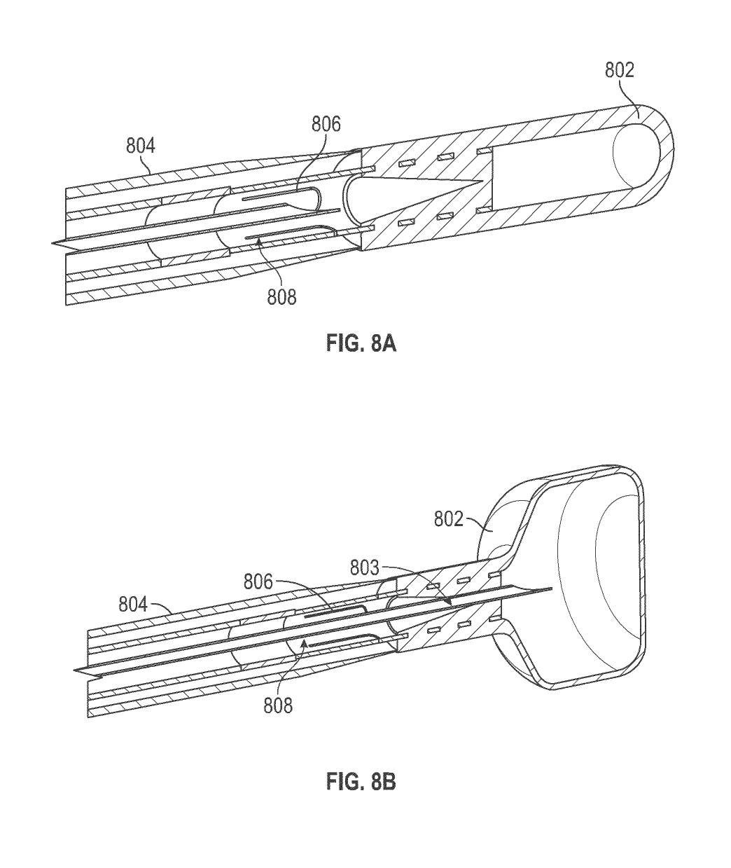

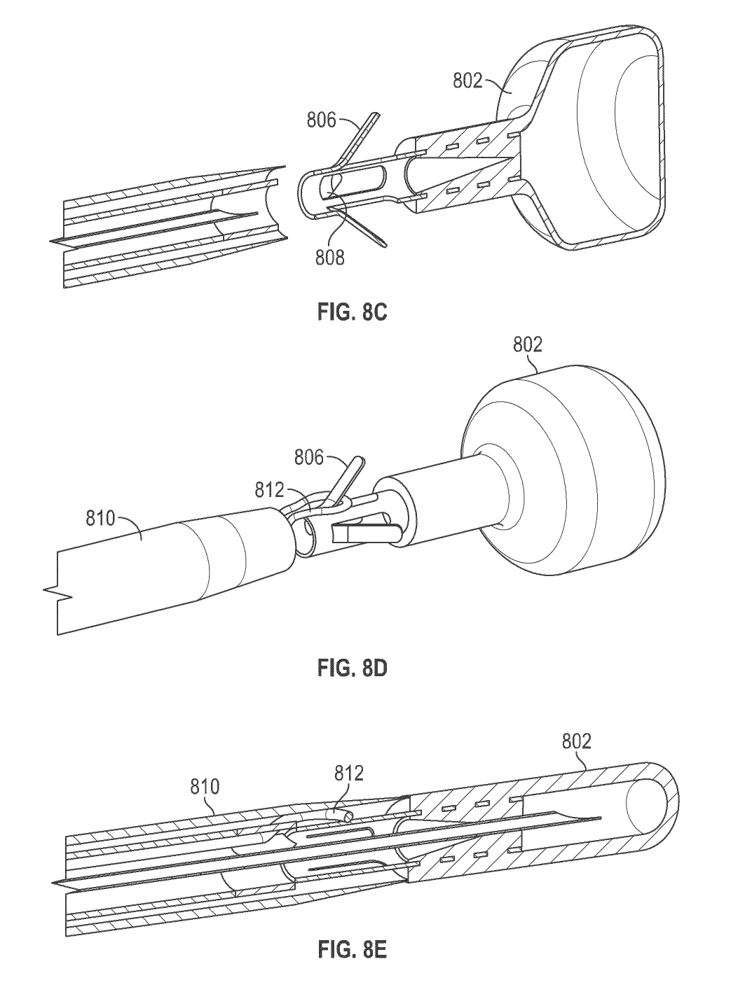

In some embodiments, the occlusion device can be an inflatable balloon, such as a silicone balloon. This configuration is shown in FIGS. 8A-E. As shown, the balloon 802 can be attached to the end of a catheter 804. Upon insertion into the proper position, the balloon 802 can be inflated to press against the walls of a blood vessel. In some embodiments, an inflation mechanism 803 can be included in the catheter 804 to inflate the balloon 802. The balloon 802 can be released into the blood vessel to occlude the vessel.

The balloon 802 can be releasably connected with the catheter 804 at a sealable tube 808 located on an open end of the balloon 802. The tube 808 can include a “finger” 806 or plurality of fingers that extend radially outwards upon release from the catheter 804 (shown in FIG. 8C). Any number of fingers 806 can be used, such as 1, 2, 3, 4, 5, or 6 fingers. In some embodiments, the fingers 806 can be spaced evenly around the circumference of the tube 808. The fingers 806 can be used for retrieval of the balloon 802. The fingers 806 can be laser cut from the tube 808 itself or can be extension located on the tube 808.

As discussed below, a catheter 810 (either the same or different than catheter 804) can be reinserted into the blood vessel with a retrieval mechanism 812 (such as a lasso or hook) to attach to at least one of the fingers 806. The balloon 802 can then be pulled back against the catheter 810 and removed. In some embodiments, the fingers 806 are configured to flex so that they are generally parallel to the catheter 810 when inside. When outside the catheter 810, the finger 806 can expand outwards. In some embodiments, the fingers 806 are angled towards the balloon 802 when in the expanded position for ease of compressing the fingers 806 into the catheter 810.

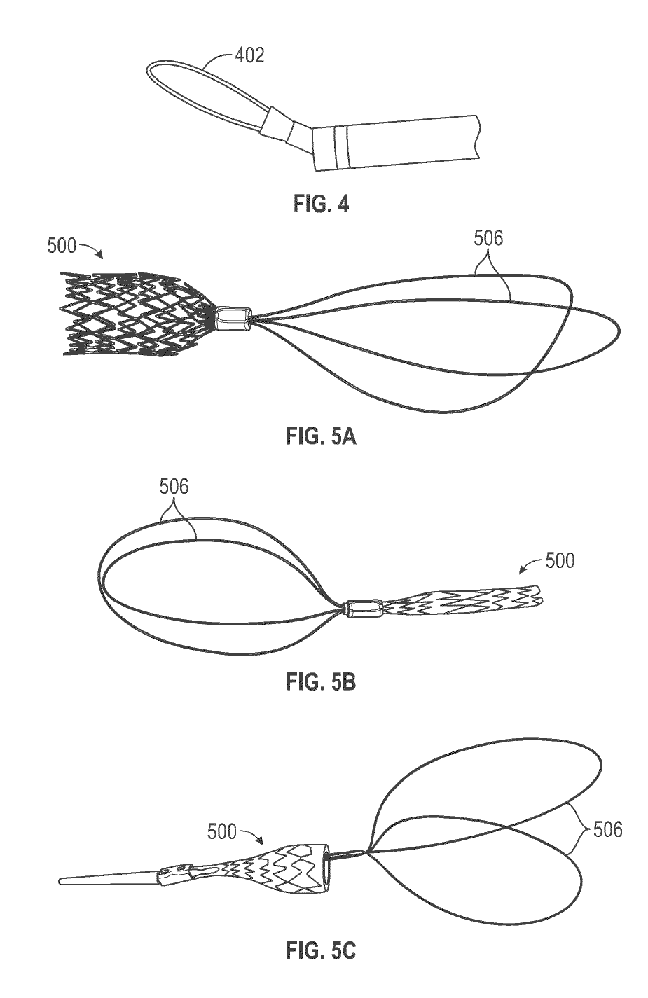

FIG. 4 illustrates an embodiment of a retrieval element that can be used to attach to the hooks/fingers described above. As shown, the retrieval element can be a loop or lasso 402. The loop 402 can be generally flexible, or can be rigid. The loop 402 can be configured to be stored within a catheter/sheath for deployment in a blood vessel and retrieval of an embodiment of an occlusion device, as discussed below.

FIGS. 5A-C illustrate another embodiment of an engagement element that can be used with an occlusion device. As shown, the occlusion device 500 can have a plurality of wire snares 506 attached to an end of the device 500. As shown, the snares 506 can have a significantly greater diameter than loop 402 of FIG. 4. The loops 506 can be generally flexible, or can be rigid. In some embodiments, the snares 506 can have a diameter that is equal or greater than the length of the occlusion device. In some embodiments, the snares 506 can be nitinol snares. When a snare 506 is used, the retrieval element can be a hook shape to engage with the snare 506. The snares 506 can be used instead of the hooks described above, or the device 500 can include both hooks and snares on opposite ends.

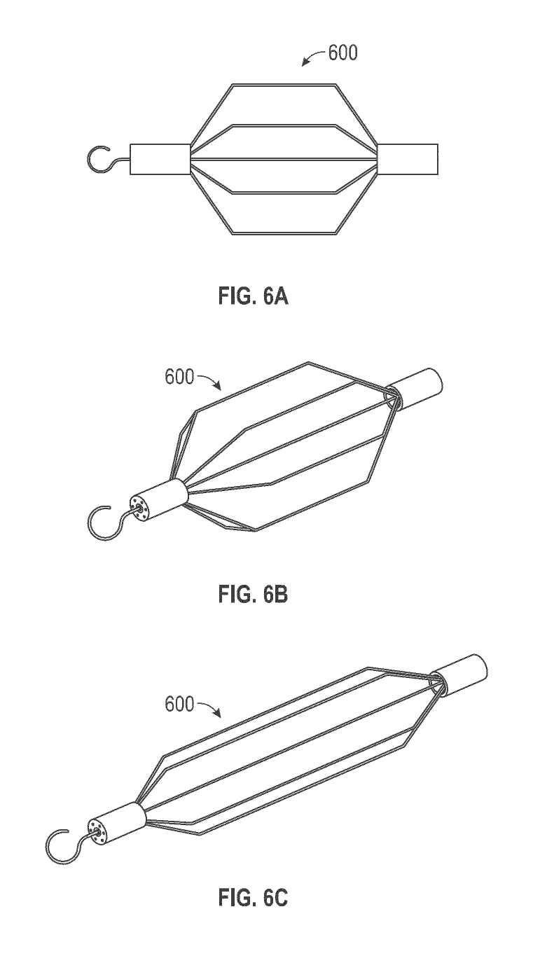

FIGS. 6A-C illustrate different configurations of embodiments of an occlusion device in which any of the above configurations can be used. FIGS. 6A-B show an expanded configuration of an occlusion device 600, while FIG. 6C shows a collapsed configuration. In the collapsed configuration, the device 600 can be retracted into a catheter or sheath (or collapsing can occur when the device is being drawn into the catheter or sheath). As shown, the collapsed configuration can have a significantly smaller cross sectional area than the expanded configuration. In some embodiments, the occlusion device 600 can be self-expanding. In some embodiments, the occlusion device 600 can be manually expanded, such as through the use of a balloon. FIGS. 6A-C show a hook engagement element 606, though other types of engagement elements can be used and the type of engagement element is not limiting, such as the other occlusion devices discussed herein. In some embodiments, the occlusion device 600 can be expanded and collapsed multiple times.

### Deployment and Recovery

Disclosed herein are example embodiments of the deployment and use of a vascular occlusion device, however other methods can be used as well. The disclosed methods could be used with any of the above configurations of occlusion devices.

The occlusion device can be inserted into the lumen of a patient, such as a blood vessel. In some embodiments, a catheter can be used to deliver the device, such as discussed above. Once there, the occlusion device can be expanded in order to block, partially block, or reduce blood flow through the lumen. For example, the occlusion device can have an expanded diameter that is the same as the internal diameter of the lumen. In some embodiments, the occlusion device can provide outward pressure on the internal diameter of the lumen. The occlusion device can be self-expanding (such as with a compressed nitinol structure) or can be manually expanded (such as by expanding a balloon through the addition of fluids). The occlusion device can be left until removal is desired.

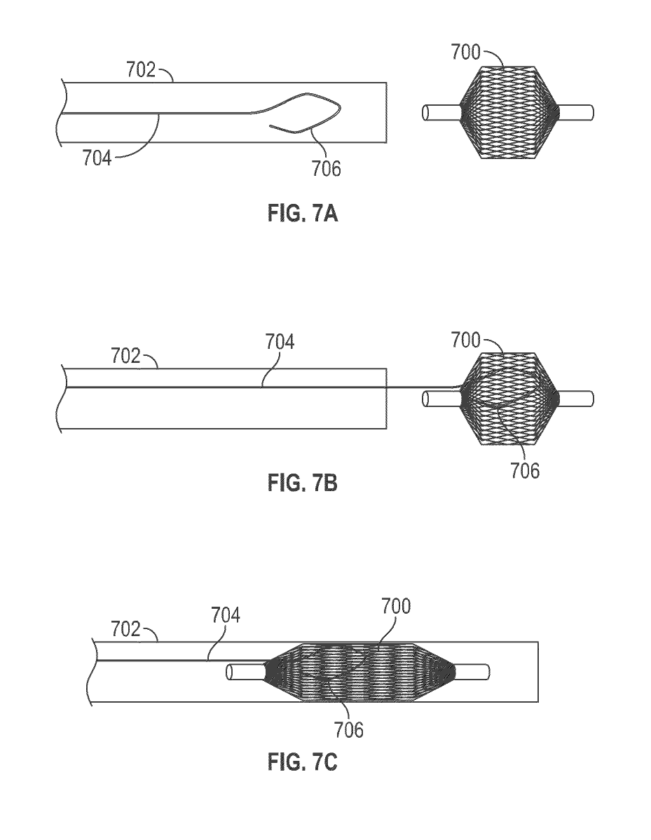

FIGS. 7A-C illustrate a general method for removing an occlusion device from a blood vessel. As shown in FIG. 7A, the occlusion device (implant) 700 can be located within a patient (for example, after self-expansion or manual expansion). A catheter/sheath 702 can then be inserted into the blood vessel near the occlusion device 700. The catheter/sheath can contain, for example, a wire 704 (e.g., re-capture device) having a hooked end 706. Different types of hooks can be used as well. In some embodiments, the hook 706 can be flexible or rigid.

As shown in FIG. 7B, the hooked wire can be driven into the body of the occlusion device 700 (such as the mesh or struts discussed in the embodiments above). This method can be used when there is no engagement element on the ends of the device 700. The occlusion device can contain a plurality of mesh/struts that are sized so that the wire 704 can be inserted into the body of the occlusion member 700, and the hooked end 706 can attach to the mesh/struts of the occlusion device 700. In some embodiments, the wire 702 can pierce any outer covering of the occlusion device 700.

In FIG. 7C, the occlusion device 700 can be retracted into the sheath 702 by pulling the wire 704 back into the sheath 702. The sheath 702 can cause the occlusion device 700 to contract, or the occlusion device 700 can be contracted prior to pulling it into the sheath 702.

In some embodiments, the hooked end 706 may be used to attach to a removal device on the occlusion device 700, such as a hook or fingers discussed above. Thus, the wire 704 would not have to pierce any coating on the device 700. However, in some embodiments the coating would cover the retrieval elements and the wire 704 could pierce the covering. Thus, in some embodiments the wire 704 can be generally co-axially engaging with the occlusion device 700.

FIG. 8A-E show another embodiment for deployment and retrieval of an occlusion device. As shown in FIG. 8A, in some embodiments the occlusion device 802 can be loaded into a delivery vehicle 804. This delivery vehicle 804 can be, for example, a large delivery catheter or sheath. While in the delivery vehicle 804, the occlusion device 802 can be in a contracted position to allow for ease of delivery.

The delivery vehicle 804 can then be introduced into the vasculature of a patient, for example into a vein or artery. Upon positioning of the vehicle 804 in a desire location, the delivery vehicle 804 can be removed from the occlusion device 802. The occlusion device 802 can then expand, either automatically upon removal of the delivery vehicle 804 or through some other method, until the occlusion device 802 partially or fully occludes the vasculature as desired. FIG. 8B illustrates an embodiment where the occlusion device 802 is inflated or self-expanded, though the method of expanding the occlusion device 802 is not limiting. After expansion, the occlusion device 802 can be released into a blood vessel, as shown in FIG. 8C. In some embodiments, the vehicle 804 can be removed from the patient. In some embodiments, the device 802 can be removed in a partially collapsed or fully collapsed position.

The occlusion device 802 can then remain in the patient for a desired time. For example, the occlusion device 802 can remain in a patient for days, months, or years.

When the occlusion device 802 is to be removed, a removal/recovery device 812 can be introduced into the patient's blood vessel. The removal/recovery device 812 can then use a recovery feature, such as the ones described above. This is shown in FIG. 8D. While FIG. 8D shows a particular embodiment of an occlusion device, it will be understood that all of the above embodiments of occlusion devices can be removed in a similar manner. The removal/recovery device 812 can be inserted into the patient in a catheter 810. Further, the occlusion device 802 can be unexpanded.

After recovery, the catheter 810 can be retraced outside of the patient, as shown in FIG. 8E. In some embodiments, the occlusion device 802 can be brought into the catheter 810 upon removal. In some embodiments, the occlusion device 802 can remain outside the catheter 810.

The occlusion device 802 can be un-expanded (e.g., collapsed) either before removal or during the removal process. For example, if the occlusion device 802 is a balloon, the balloon can be punctured, thereby allowing any fluid inside to escape. Thus, the collapsed occlusion device 802 can be withdrawn into the catheter 810. The puncturing can occur using an instrument in the catheter 810, such as wire 704, or a separate instrument. If the occlusion device 802 is self-expanding, the catheter 810 can have a smaller diameter than the occlusion device 802 and when the occlusion device 802 is drawn into the catheter 810 it will be collapsed.

It will be understood that components of the different occlusion devices can be combined with one another to form different embodiments of occlusion devices. For example, the fingers shown in FIG. 8C could be used instead of the hook of FIG. 1B, and vice-versa. Thus, the disclosed retrieval elements can be used for any number of different embodiments of the occlusion devices, and not just the occlusion devices that they are described in particularly with.

### Anchoring Element

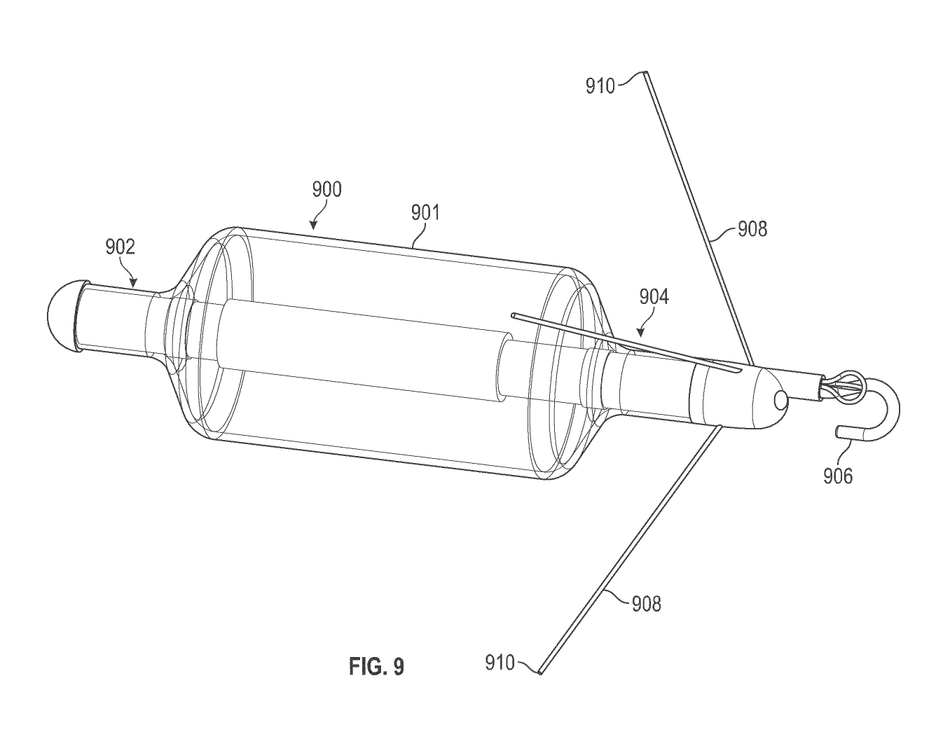

FIG. 9 illustrates an embodiment of an occlusion device 900, similar to those described above, having a first end 902 and a second end 904 and a hook attachment element 906 at the second end 904. Further, the occlusion device 900 can have a plurality of anchoring elements 908. While FIG. 9 illustrates three anchoring elements 908, other numbers of anchoring elements could be used as well, such as 1, 2, 3, 4, 5, 6, 7, 8, 9, or 10 anchoring elements.

As shown, the anchoring elements 908 can extend outwardly away from the occlusion device 900 while being angled towards the first end 902 of the occlusion device 900. The anchoring elements 908 can extend farther than a maximum diameter of the body 901 of the occlusion device 900 in its fully expanded position.

The anchoring elements 908 can be used to prevent migration of the occlusion device 900 in a patient's blood vessel. In particular, if a balloon is used, such as a silicon balloon, and is over pressurized against the blood vessel, the balloon may deflate over time, releasing the occlusion device form the patient's blood vessel. However, the anchoring elements 908 can be used instead, allowing for the balloon to have only ambient pressure, and reducing the risk of the occlusion device releasing in the blood vessel. In particular, ends 910 of the anchoring elements 908 can abut against the blood vessel wall (e.g., engage with the blood vessel wall), preventing motion. In some embodiments, the anchoring elements 908 can be directed generally in the direction of blood flow. In some embodiments, the anchoring elements 908 can be directed generally in the direction against blood flow.

From the foregoing description, it will be appreciated that an inventive product and approaches for a removable occlusion device are disclosed. While several components, techniques and aspects have been described with a certain degree of particularity, it is manifest that many changes can be made in the specific designs, constructions and methodology herein above described without departing from the spirit and scope of this disclosure.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount.

Some embodiments have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. An occlusion device for reversibly occluding a body lumen, the occlusion device comprising:
an elongate member, comprising first and second end portions, wherein the elongate member is configured to reversibly expand from a collapsed state to an expanded state at a site within the body lumen, and wherein the elongate member is further configured to at least partially occlude the body lumen at the site; and
a retrieval element coupled to the elongate member along one of the first or second end portions and extending generally outward from the elongate member into the body lumen.

2. The occlusion device of claim 1, wherein one or both of the first and second end portions are closed.

3. The occlusion device of claim 1, wherein the body lumen is a blood vessel.

4. The occlusion device of claim 1, wherein the retrieval element comprises a hook.

5. The occlusion device of claim 1, wherein the retrieval element comprises at least one loop.

6. The occlusion device of claim 1, wherein the retrieval element comprises at least one expandable finger.

7. The occlusion device of claim 1, wherein the elongate member comprises a silicone balloon.

8. The occlusion device of claim 1, wherein the elongate member comprises a mesh plug.

9. The occlusion device of claim 1, wherein the elongate member comprises a stent.

10. The occlusion device of claim 1, further comprising a covering configured to at least partially surround the elongate member.

11. The occlusion device of claim 10, wherein the covering comprises PTFE, FEP, very high density ePTFE, or silicone.

12. The occlusion device of claim 1, further comprising at least one anchoring element configured to extend radially outward from the elongate member toward a wall of the body lumen and engage the wall.

13. An occlusion system comprising:
an occlusion device comprising:
an elongate member, comprising first and second end portions, wherein the elongate member is configured to reversibly expand from a collapsed state to an expanded state at a site within a body lumen, and wherein the elongate member is further configured to at least partially occlude the body lumen at the site; and
a retrieval element coupled to the elongate member along one of the first or second end portions and extending generally outward from the elongate member into the body lumen;
a re-capture device configured to engage the retrieval element of the occlusion device; and
a sheath configured to surround the re-capture device together with the engaged occlusion device, wherein the elongate member of the occlusion device is retained in the collapsed state within the sheath..

14. A method of reversibly occluding a blood vessel for a period of time, the method comprising:
deploying an occlusion device at a site within the blood vessel of a patient, the occlusion device comprising:
an elongate member, comprising first and second end portions, wherein the elongate member is configured to reversibly expand from a collapsed state to an expanded state at the site within the blood vessel, and wherein the elongate member is further configured to occlude the blood vessel at the site; and
a retrieval element coupled to the elongate member along one of the first or second end portions and extending generally outward from the elongate member into a lumen of the blood vessel;
expanding the elongate member, thereby occluding the blood vessel at the site;
collapsing the elongate member after the period of time has passed;
deploying a re-capture device at the site within the blood vessel, wherein the re-capture device is configured to engage the retrieval element;
engaging the retrieval element;
co-axially engaging the re-capture device together with the engaged occlusion device within a sheath; and
removing the sheath with the re-capture device and engaged occlusion device from the patient.

15. The method of claim 14, wherein the occlusion device self-expands when deployed at the site.

16. The method of claim 14, wherein the deploying the re-capture device comprises inserting the re-capture device at least partially surrounded by a second sheath.

17. The method of claim 14, wherein the collapsing occurs when the elongate member is pulled into the sheath.

18. The method of claim 14, wherein the collapsing occurs by puncturing the elongate member.

19. A method of inducing weight loss, the method comprising:
deploying an occlusion device at a site within a target blood vessel that supplies blood to an organ of the gastrointestinal tract of a patient, the occlusion device comprising:
an elongate member, comprising first and second end portions, wherein the elongate member is configured to reversibly expand from a collapsed state to an expanded state at the site, and wherein the elongate member is further configured to at least partially occlude the target blood vessel at the site; and
a retrieval element coupled to the elongate member along one of the first or second end portions and extending generally outward from the elongate member into a lumen of the target blood vessel; and
expanding the elongate member, thereby at least partially occluding the target blood vessel, restricting blood flow to the organ, and inducing weight loss.

20. The method of claim 19, wherein the target blood vessel is a celiac artery.

21. The method of claim 19, wherein the target blood vessel is selected from the group consisting of the left gastric artery, the right gastric artery, the left gastroepiploic artery, the right gastroepiploic artery, or the common hepatic artery.
